(19) United States
(12) Patent Application Publication
(10) Pub. No.: US 2016/0310106 A1
(43) Pub. Date: Oct. 27, 2016

(54) NONINVASIVE IMAGING APPARATUS FOR GASTROINTESTINAL TRACK

(71) Applicant: POSTECH ACADEMY-INDUSTRY FOUNDATION, Pohang-si (KR)

(72) Inventors: Chulhong KIM, Pohang-si (KR); Mansik JEON, Daegu-si (KR); Changho Lee, Daegu-si (KR); Jeesu Kim, Seoul (KR)

(21) Appl. No.: 15/134,732

(22) Filed: Apr. 21, 2016

(30) Foreign Application Priority Data
Apr. 23, 2015 (KR) 10-2015-0057226

(51) Int. Cl. A61B 8/08; A61B 8/00

(52) U.S. Cl. CPC A61B 8/481; A61B 8/5207; A61B 8/4483; A61B 8/08; A61B 8/54; A61B 5/0095

(57) ABSTRACT

Provided is a non-invasive imaging apparatus of acquiring an image of a region of interest of an object, including: a laser supplying a laser beam; a fiber bundle transmitting the laser beam to the region of interest of the object; a transducer detecting an ultrasound signal output from the region of interest of the object corresponding to the laser beam supplied from the fiber bundle and generating and outputting an ultrasound detection signal; and an ultrasound device generating and outputting a photoacoustic image by using the ultrasound detection signal output from the transducer, wherein the region of interest of the object is a gastrointestinal track of an animal, wherein the transducer detects the ultrasound signal of the gastrointestinal track of the animal into which a contrast agent is injected, and wherein the photoacoustic image is an image of the region of interest into which the contrast agent is injected.

### CROSS-REFERENCE TO RELATED PATENT APPLICATION

This application claims the benefit of Korean Patent Application No. 10-2015-0057226, filed on Apr. 23, 2015, in the Korean Intellectual Property Office, the disclosure of which is incorporated herein in its entirety by reference.

### BACKGROUND OF THE INVENTION

1. Field of the Invention

The present invention relates to non-invasive imaging technology, and more particularly, to a non-invasive imaging apparatus for a gastrointestinal track capable of acquiring a photoacoustic image or selectively acquiring a photoacoustic image or an ultrasound image of the gastrointestinal track of an animal according to time by using a contrast agent of a naphthalocyanine nano structure which is not absorbed by the gastrointestinal track but excreted from the body.

2. Description of the Related Art

The diseases most often treated in the outpatient care are diseases of gastrointestinal tracks. Images of the gastrointestinal tracks can provide great aid to diagnosis of the diseases of the gastrointestinal track s. Therefore, imaging technologies using a capsule endoscope, colonoscopy, MRI, CT, X-RAY, ultrasound imaging, and the like have been used for imaging the gastrointestinal tracks.

However, with respect to functional imaging for peristaltic motion of intestines, the intestines is hard to access, efficiency of the imaging is low, and it is difficult to non-invasively obtain images. Many studies have not been conducted on the functional imaging for peristaltic motion of intestines.

In general, disorders in intestine motion causes overgrowth of bacteria in the intestines, irritable bowel syndrome, inflammatory bowel diseases, constipation, and the like. In addition, irregular intestine motion may lead to serious side effects such as thyroid disorders, diabetes, Parkinson's disease, or the like. In order to perform accurate diagnosis and treatment of these diseases, the state of the intestine motion needs to be known.

However, methods in the related art are based on many times of trial and error. In addition, in clinical studies, generally, the process of intestine motion is measured ex vivo. Therefore, if structural and functional images of the intestines are stably and non-invasively, these images can be aid to better diagnosis and treatment of the intestine diseases.

### SUMMARY OF THE INVENTION

The present invention is to provide a non-invasive imaging apparatus for a gastrointestinal track of acquiring a photoacoustic image of the gastrointestinal track according to time by using a contrast agent of a naphthalocyanine nano structure which is not absorbed by the gastrointestinal track but excreted from a body.

The present invention is also to provide a non-invasive imaging apparatus for a gastrointestinal track configured to selectively provide one of an ultrasound image and a photoacoustic image of the gastrointestinal track of an animal.

According to a first aspect of the present invention, there is provided a non-invasive imaging apparatus of acquiring an image of a region of interest of an object, including: a laser which supplies a laser beam; a fiber bundle which transmits the laser beam to the region of interest of the object; a transducer which detects an ultrasound signal output from the region of interest of the object corresponding to the laser beam supplied from the fiber bundle and generates and outputs an ultrasound detection signal; and an ultrasound device which generates a photoacoustic image by using the ultrasound detection signal output from the transducer and outputs the photoacoustic image, wherein the region of interest of the object is a gastrointestinal track of an animal, wherein the transducer detects the ultrasound signal of the gastrointestinal track of the animal into which a contrast agent is injected, and wherein the photoacoustic image is an image of the region of interest into which the contrast agent is injected.

According to a second aspect of the present invention, there is provided a non-invasive imaging apparatus of acquiring an image of a region of interest of an object, including: a laser which supplies a laser beam; a fiber bundle which transmits the laser beam to the region of interest of the object; a transducer which detects an ultrasound signal output from the region of interest of the object corresponding to the laser beam supplied from the fiber bundle and generates and outputs an ultrasound detection signal; an ultrasound device which generates a photoacoustic image by using the ultrasound detection signal output from the transducer and outputs the photoacoustic image; and a control device which controls the laser and the ultrasound device according to a control command signal input externally to generate and output one of an ultrasound image and a photoacoustic image, wherein, if a first control command signal requiring the ultrasound image is input, the control device controls the ultrasound device to allow the transducer to transmit an ultrasound signal to the region of interest of the object and receive the ultrasound signal returned from the object, and the ultrasound device generates and outputs the ultrasound image by using the ultrasound detection signal received from the transducer corresponding to the ultrasound signal, and wherein, if a second control command signal requiring the photoacoustic image is input, the control device drives the laser to irradiate the region of interest of the object with the laser beam, the transducer detects the ultrasound signal output from the region of interest of the object according to the laser beam, and the ultrasound device generates and outputs the photoacoustic image by using the ultrasound detection signal received from the transducer.

According to the present invention, it is possible to obtain effects that it is possible to non-invasively acquire a photoacoustic image or an ultrasound image of a gastrointestinal track of an animal by using a contrast agent of a naphthalocyanine nano structure which is not absorbed by the gastrointestinal track but excreted from a body and it is possible to allow a motion of the gastrointestinal track according to time to be studied.

According to the present invention, since the contrast agent is not accumulated in the body but excreted from the body, it is possible to obtain an effect that it is possible to prevent potential side effects of a contrast agent in advance.

### BRIEF DESCRIPTION OF THE DRAWINGS

The patent or application file contains at least one drawing executed in color. Copies of this patent or patent application publication with color drawing (s) will be provided by the Office upon request and payment of the necessary fee.

FIGS. 1A to 1C are diagrams illustrating a degree of excretion of a contrast agent in intestines which is a gastrointestinal track and in excreta;

FIG. 2 is a diagram illustrating a configuration of a non-invasive imaging apparatus according to a first embodiment of the present invention;

FIG. 3 is a diagram illustrating a configuration of a non-invasive imaging apparatus according to a second embodiment of the present invention; and

FIGS. 4A to 4D are diagrams illustrating an example of in-vivo photoacoustic images acquired from a gastrointestinal track and intestines of a BALB/c mouse by using a naphthalocyanine contrast agent according to the present invention.

### DETAILED DESCRIPTION OF THE INVENTION

Photoacoustic imaging technology is a non-ionization-type imaging method having deep propagation depth. This method can be potentially combined with inexpensive, small-sized ultrasound devices, and thus, this method is a non-invasive imaging method capable of safely diagnosing a state of the gastrointestinal track such as an intestine motion. Particularly, photoacoustic imaging is useful to imaging using a near IR contrast agent.

Therefore, in the present invention, while being flowed along the gastrointestinal track, a naphthalocyanine nano structure which is neither decomposed nor absorbed is used to non-invasively acquire real-time photoacoustic image data of the gastrointestinal track.

Now, the naphthalocyanine nano structure is described more in detail.

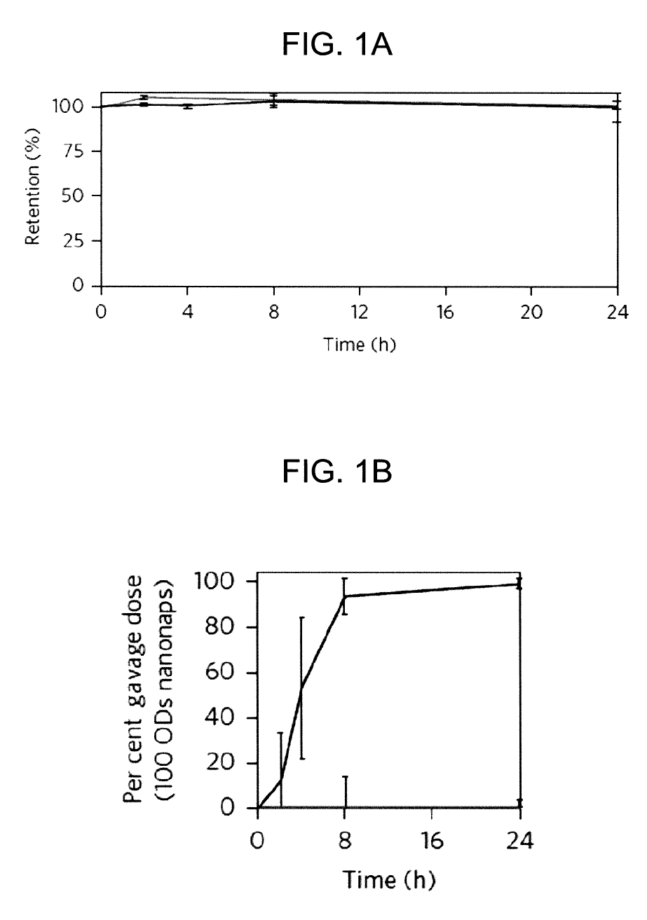

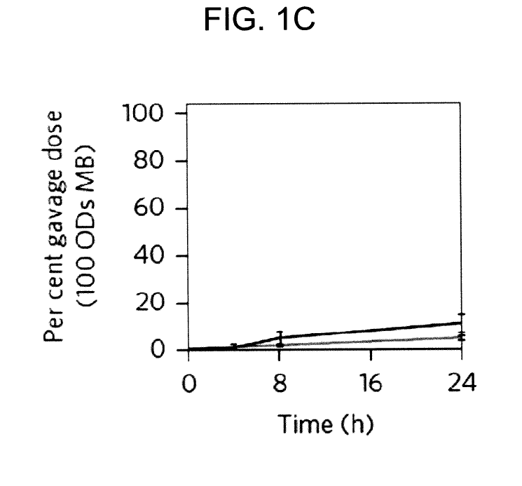

FIGS. 1A to 1C are diagrams illustrating a degree of excretion of a contrast agent in intestines which is a gastrointestinal track and in excreta. FIG. 1A illustrates degrees of detection of the naphthalocyanine nano structure in the gastrointestinal track (red) and intestines (black). FIG. 1B illustrates degrees of detection of the naphthalocyanine nano structure in feces (black) and urine (red). FIG. 1C illustrates degrees of detection of the methylene blue in feces (black) and urine (red).

Referring to FIGS. 1A to 1C, the naphthalocyanine nano structure is detected in all of the gastrointestinal tracks and the intestines. The contrast agent is not detected in the urine, but most of the contrast agent is detected in the feces. Particularly, in comparison with methylene blue as the contrast agent reprehensively used in the photoacoustic imaging technology, a larger amount of the naphthalocyanine nano structure is detected in the urine and feces, in other words, a larger amount of the naphthalocyanine nano structure is excreted from the body. Accordingly, it can be understood that the naphthalocyanine contrast agent is effective to photoacoustic imaging of the gastrointestinal track and the intestines and, since most amount of the naphthalocyanine contrast agent is excreted from the body, the naphthalocyanine contrast agent is not accumulated in the body.

### First Embodiment

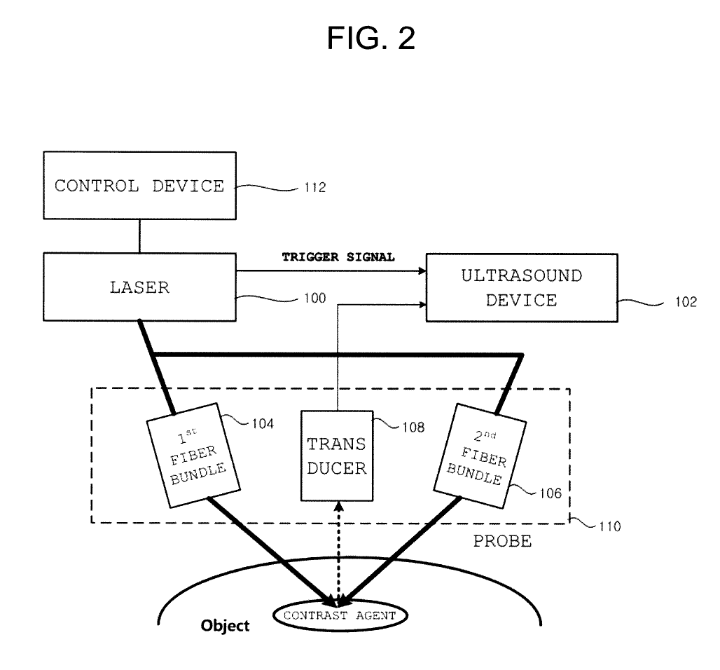

Now, a non-invasive imaging apparatus for a gastrointestinal track of an animal as a region of interest of an object by using a naphthalocyanine nano structure according to a first embodiment of the present invention will be described with reference to FIG. 2.

FIG. 2 is a diagram illustrating a configuration of the non-invasive imaging apparatus according to the first embodiment of the present invention. Referring to FIG. 2, the non-invasive imaging apparatus according to the first embodiment of the present invention is configured to include a laser 100, an ultrasound device 102, first and second fiber bundles 104 and 106, a transducer 108, and a control device 112. Herein, a probe 110 is configured so that the transducer 108 and the first and second fiber bundles 104 and 106 are accommodated into one housing.

The laser 100 may be configured with a fiber bundle laser. Particularly, the laser may be configured with one movable laser system obtained by combining an ND: YAG pump laser and a tunable OPO laser. The laser 100 generates and splits a laser beam and supplies the laser beams to the first and second fiber bundles 104 and 106.

In addition, the laser generates a trigger signal for synchronization with the ultrasound device 102 and supplies the trigger signal to the ultrasound device 102. Particularly, output wavelength and output intensity of the laser beam of the laser 100 are controlled by software installed in the control device 112.

The ultrasound device 102 receives ultrasound detection signal detected by the transducer 108 to generate photoacoustic image data. Particularly, the ultrasound device 102 acquires photoacoustic image data in synchronization with the trigger signal supplied by the laser 100 to generate a photoacoustic image.

The probe 110 is configured so that the first and second fiber bundles 104 and 106 and the transducer 108 are accommodated in one housing. Therefore, the first and second fiber bundles 104 and 106 and the transducer 108 can be manipulated by using one hand.

The first and second fiber bundles 104 and 106 are disposed to be separated from each other, and the transducer 108 is disposed between the first and second fiber bundles 104 and 106.

The first and second fiber bundles 104 and 106 receives the laser beam emitted from the laser 100 and supply the laser beam to a specific position of the region of interest of the object facing the user's hand.

The transducer 108 detects an ultrasound signal generated when the object absorbs the laser beam irradiated from the first and second fiber bundles 104 and 106 to thermo-elastically expand, and the transducer supplies an ultrasound detection signal to the ultrasound device 102.

The region of interest of the object is a gastrointestinal track, intestines, or the like which contains the naphthalocyanine contrast agent according to the present invention.

In the above-described first embodiment of the present invention, after the gastrointestinal track, the intestines, or the like is allowed to contain the contrast agent having a naphthalocyanine nano structure, photoacoustic imaging is performed while the probe 110 is being moved to track the flow of the naphthalocyanine nano structure through the gastrointestinal track and the intestines, so that structural and functional image data of the intestines can be acquired.

### Second Embodiment

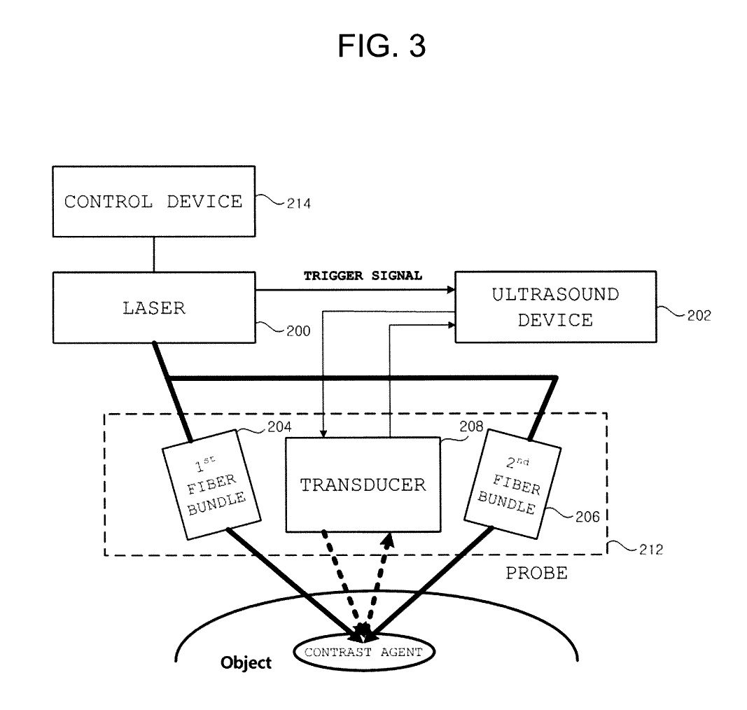

Now, a non-invasive imaging apparatus using a naphthalocyanine nano structure according to a second embodiment of the present invention will be described with reference to FIG. 3. FIG. 3 is a diagram illustrating a configuration of the non-invasive imaging apparatus according to the second embodiment of the present invention.

Referring to 3, the non-invasive imaging apparatus according to the second embodiment of the present invention is configured to include a control device 214, a laser 200, an ultrasound device 202, first and second fiber bundles 204 and 206, and a transducer 208. Herein, a probe 212 is configured so that the transducer 208 and the first and second fiber bundles 204 and 206 are accommodated into single housing.

The second non-invasive imaging apparatus for gastrointestinal track is configured to include the laser 200, the ultrasound device 202, and the probe 212.

The laser 200 may be configured with a fiber bundle laser. The laser may be configured with one movable laser system obtained by combining an ND: YAG pump laser and a tunable OPO laser. The laser 200 generates and splits a laser beam and supplies the laser beams to the first and second fiber bundles 204 and 206. In addition, the laser generates a trigger signal for synchronization with the ultrasound device 202 and supplies the trigger signal to the ultrasound device 202. Particularly, output wavelength and output intensity of the laser beam of the laser 200 are controlled by control software installed in the control device 214.

The ultrasound device 202 drives the transducer 208 and receives ultrasound detection information received by the transducer 208 to generate ultrasound image data.

The control device 214 is configured to control the ultrasound device according to a control command input from an operator or the like to output selectively one of a photoacoustic image and an ultrasound image of the region of interest of the object.

If a first control command signal requiring an ultrasound image is input externally, the control device 214 drives the non-invasive imaging apparatus in an ultrasound imaging mode; and if a second control command signal requiring a photoacoustic image is input externally, the control device drives the non-invasive imaging apparatus in a photoacoustic imaging mode.

In the ultrasound imaging mode, the control device controls the ultrasound device 202 to allow the transducer 208 to transmit an ultrasound signal to the region of interest of the object and receive the ultrasound signal returned from the object. The transducer detects an ultrasound signal returned from the object and provides an ultrasound detection signal corresponding to the returned ultrasound signal to the ultrasound device. The ultrasound device 202 generates an ultrasound image by using the ultrasound detection signal received from the transducer 208.

On the other hand, in the photoacoustic imaging mode, the control device drives the laser to emit the laser beam to the region of interest of the object and, simultaneously, controls the laser to supply a trigger signal to the ultrasound device. The ultrasound device 202 drives the transducer 208 according to the trigger signal to detect a photoacoustic ultrasound signal generated from the region of interest of the object corresponding to the laser beam. The ultrasound device 202 receives the detected photoacoustic ultrasound signal from the transducer. The ultrasound device 202 receives the photoacoustic ultrasound signal from the transducer 208 and generates and outputs a photoacoustic image. Particularly, the ultrasound device 202 generates the photoacoustic image data in synchronization with the trigger signal supplied from the second fiber bundle laser 200.

The probe 212 is configured with the first and second fiber bundles 204 and 206 and the transducer 208 which are accommodated into one housing. Therefore, the first and second fiber bundles 204 and 206 and the transducer 208 can be manipulated with one hand. The first and second fiber bundles 204 and 206 are disposed to be separated from each other, and the transducer 208 is disposed between the first and second fiber bundles 204 and 206.

The first and second fiber bundles 204 and 206 receive the laser beam emitted from the laser 200 and supply the laser beam to a specific position of an animal facing the user's hand.

The region of interest of the object is a gastrointestinal track, intestines, or the like which contains the naphthalocyanine contrast agent according to the present invention.

In the above-described second embodiment of the present invention, after the gastrointestinal track, the intestines, or the like is allowed to contain the contrast agent having a naphthalocyanine nano structure, a photoacoustic image or an ultrasound image is selectively acquired while the probe 212 is being moved to track the flow of the naphthalocyanine nano structure through the gastrointestinal track and the intestines, so that structural and functional image data of the intestines can be acquired.

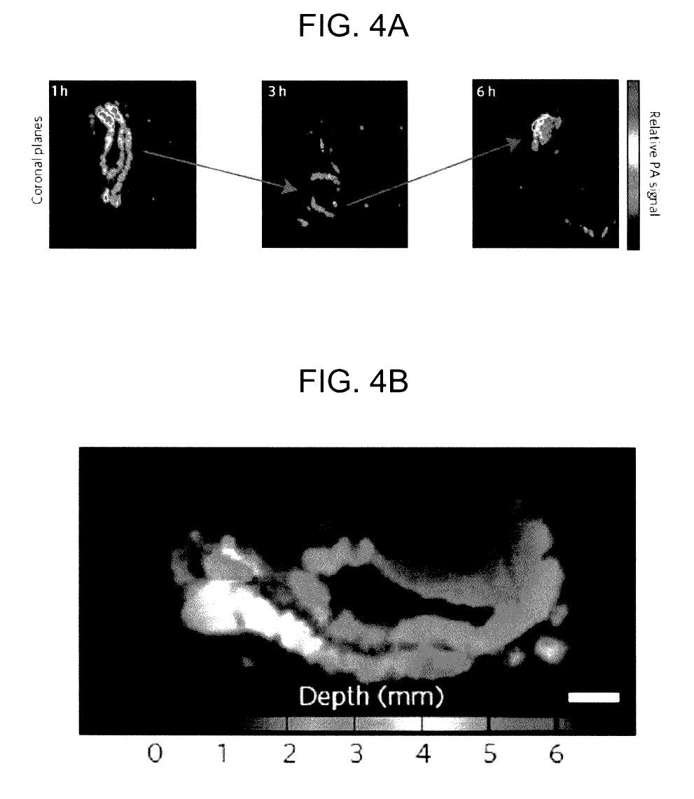

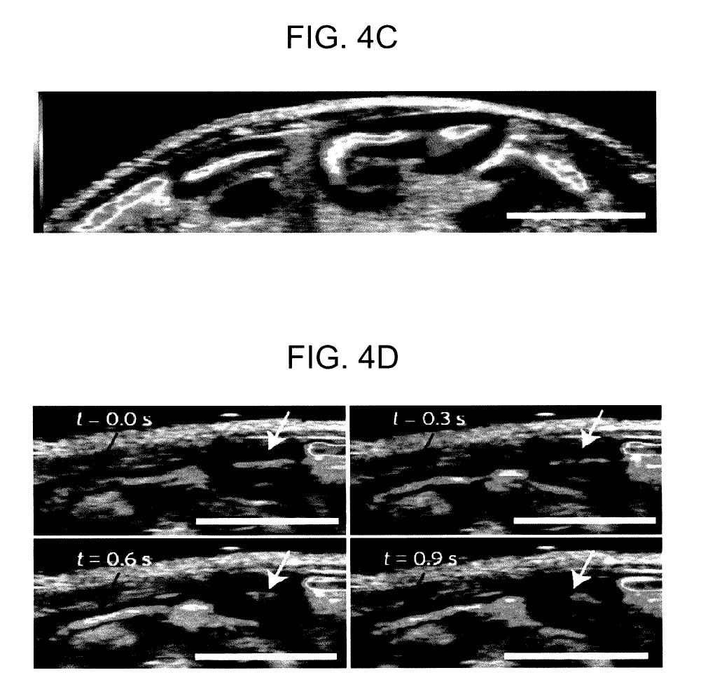

FIGS. 4A to 4D are diagrams illustrating an example of in-vivo photoacoustic images acquired from a gastrointestinal track and intestines of a BALB/c mouse by using a naphthalocyanine contrast agent according to the present invention. FIG. 4A illustrates a flow of the naphthalocyanine contrast agent along the intestines according to time. FIG. 4B illustrates the photoacoustic image of the intestines with different colors according to the depth of the signal. FIG. 4C illustrates a result of imaging of the flow of the naphthalocyanine contrast agent in real-time by combining the ultrasound image and the photoacoustic image. FIG. 4D illustrates a result of analysis of an inner portion of the intestines from the image. The motion of intestines can be seen by observing the flow of the naphthalocyanine contrast agent in inner portions of the intestines. In the figure, black arrows indicate inflow, and white arrows indicate outflow.

What is claimed is:

1. Anon-invasive imaging apparatus of acquiring an image of a region of interest of an object, comprising:
a laser which supplies a laser beam;
a fiber bundle which transmits the laser beam to the region of interest of the object;
a transducer which detects an ultrasound signal output from the region of interest of the object corresponding to the laser beam supplied from the fiber bundle and generates and outputs an ultrasound detection signal according to the detected ultrasound signal; and
an ultrasound device which generates a photoacoustic image by using the ultrasound detection signal output from the transducer and outputs the photoacoustic image,
wherein the region of interest of the object is a gastrointestinal track of an animal,
wherein the transducer detects the ultrasound signal of the gastrointestinal track of the animal into which a contrast agent is injected, and
wherein the photoacoustic image is an image of the region of interest into which the contrast agent is injected.

2. The non-invasive imaging apparatus according to claim 1, wherein the contrast agent is a naphthalocyanine nano structure.

3. The non-invasive imaging apparatus according to claim 1,
wherein the fiber bundle is configured with first and second fiber bundles,
wherein the laser beam supplied from the laser is split into two laser beams, and the two laser beams are supplied to the respective first and second fiber bundles, and
wherein the first and second fiber bundles irradiate the region of interest of the object with the laser beams.

4. The non-invasive imaging apparatus according to claim 1,
wherein the laser supplies the laser beam to the region of interest of the object and, simultaneously, supplies a trigger signal to the ultrasound device, and
wherein the ultrasound device receives the ultrasound detection signal from the transducer according to the trigger signal and generates the photoacoustic image by using the received ultrasound detection signal.

5. The non-invasive imaging apparatus according to claim 1, further comprising a control device,
wherein the control device controls a movement of the fiber bundle and the transducer corresponding to a flow of the contrast agent existing in the gastrointestinal track of the animal which is the region of interest of the object, so that the transducer acquires the ultrasound signal at a position where the contrast agent exists.

6. The non-invasive imaging apparatus according to claim 1, wherein the ultrasound device allows the transducer to transmit an ultrasound signal to the region of interest of the object and receive the ultrasound signal returned from the object, generates and outputs the ultrasound image by using the ultrasound detection signal provided from the transducer corresponding to the ultrasound signal.

7. The non-invasive imaging apparatus according to claim 1, further comprising a control device,
wherein, if a first control command signal requiring the ultrasound image is input, the control device controls the ultrasound device to allow the transducer to transmit the ultrasound signal to the region of interest of the object and receive the ultrasound signal returned from the object, and the ultrasound device generates and outputs the ultrasound image by using the ultrasound detection signal received from the transducer corresponding to the ultrasound signal, and
wherein, if a second control command signal requiring the photoacoustic image is input, the control device drives the laser to irradiate the region of interest of the object with the laser beam, the transducer detects the ultrasound signal output from the region of interest of the object according to the laser beam, and the ultrasound device generates and outputs the photoacoustic image by using the ultrasound detection signal received from the transducer.

8. The non-invasive imaging apparatus according to claim 1, wherein wavelength and intensity of the laser beam of the laser are variable according to a command input externally.

9. Anon-invasive imaging apparatus of acquiring an image of a region of interest of an object, comprising:
a laser which supplies a laser beam;
a fiber bundle which transmits the laser beam to the region of interest of the object;
a transducer which detects an ultrasound signal output from the region of interest of the object corresponding to the laser beam supplied from the fiber bundle and generates and outputs an ultrasound detection signal;
an ultrasound device which generates a photoacoustic image by using the ultrasound detection signal output from the transducer and outputs the photoacoustic image; and
a control device which controls the laser and the ultrasound device according to a control command signal input externally to generate and output one of an ultrasound image and a photoacoustic image,
wherein, if a first control command signal requiring the ultrasound image is input, the control device controls the ultrasound device to allow the transducer to transmit the ultrasound signal to the region of interest of the object and receive the ultrasound signal returned from the object, and the ultrasound device generates and outputs the ultrasound image by using the ultrasound detection signal received from the transducer corresponding to the ultrasound signal, and
wherein, if a second control command signal requiring the photoacoustic image is input, the control device drives the laser to irradiate the region of interest of the object with the laser beam, the transducer detects the ultrasound signal output from the region of interest of the object according to the laser beam, and the ultrasound device generates and outputs the photoacoustic image by using the ultrasound detection signal received from the transducer.

10. The non-invasive imaging apparatus according to claim 9,
wherein the region of interest of the object is a gastrointestinal track of an animal,
wherein, if the second control command signal requiring the photoacoustic image is input, the control device controls the transducer to detect the ultrasound signal of the gastrointestinal track of the animal into which the contrast agent is injected, and
wherein the photoacoustic image is the photoacoustic image of the region of interest into which the contrast agent is injected.

11. The non-invasive imaging apparatus according to claim 10, wherein the contrast agent is a naphthalocyanine nano structure.

12. The non-invasive imaging apparatus according to claim 9,
wherein the fiber bundle is configured with first and second fiber bundles,
wherein the laser beam supplied from the laser is split into two laser beams, and the two laser beams are supplied to the respective first and second fiber bundles, and
wherein the first and second fiber bundles irradiate the region of interest of the object with the laser beams.

13. The non-invasive imaging apparatus according to claim 9,
wherein the laser supplies the laser beam to the region of interest of the object and, simultaneously, supplies a trigger signal to the ultrasound device according to control of the control device, and
wherein the ultrasound device receives the ultrasound detection signal from the transducer according to the trigger signal and generates the photoacoustic image by using the received ultrasound detection signal.

14. The non-invasive imaging apparatus according to claim 10, wherein the control device controls the movement of the fiber bundle and the transducer corresponding to a flow of the contrast agent existing in the gastrointestinal track of the animal which is the region of interest of the object, so that the transducer acquires the ultrasound signal at a position where the contrast agent exists.
